(19) United States
(12) Patent Application Publication
(10) Pub. No.: US 2022/0296740 A1
(43) Pub. Date: Sep. 22, 2022

(54) SANITIZATION SYSTEMS AND METHODS FOR MEDICAL DEVICES

(71) Applicant: The Nemours Foundation, Jacksonville, FL (US)

(72) Inventor: Susan Marron, Exton, PA (US)

(21) Appl. No.: 17/696,005

(22) Filed: Mar. 16, 2022

Related U.S. Application Data
(60) Provisional application No. 63/162,187, filed on Mar. 17, 2021.

(51) Int. Cl. A61L 2/07; A61L 2/26; A47L 19/04; A47L 17/02

(52) U.S. Cl. CPC A61L 2/07; A61L 2/26; A47L 19/04; A47L 17/02; A61L 2202/122; A61L 2202/23; A61L 2202/17

(57) ABSTRACT

Systems and methods for sanitizing medical devices are provided. A system includes a container having a sanitization chamber, and the sanitization chamber is configured to receive a liquid and a medical device. A cover is configured to be supported by the container and obscure the sanitization chamber. The cover and the container are configured to be positioned in a microwave and facilitate converting the liquid to steam upon receipt of microwave energy from the microwave, and the steam sanitizes the medical device. A dryer rack is coupled to the cover, and the dryer rack is configured to support and thereby facilitates drying the medical device.

### CROSS REFERENCE TO RELATED APPLICATION

This application is related to and claims priority from U.S. Provisional Application No. 63/162,187, filed Mar. 17, 2021, the disclosure of which is incorporated herein by reference in its entirety.

### FIELD OF THE DISCLOSURE

The present disclosure generally relates to systems and methods for sanitizing medical devices. More specifically, the present disclosure relates to sanitization systems and methods for sanitizing breast pump components, baby bottles and reusable feeding equipment, or continuous positive airway pressure (CPAP) therapy components.

### BACKGROUND OF THE DISCLOSURE

Breast pump components that come into contact with human breast milk, including breast shields, bottles, connectors, membrane valves, and the like, should be cleaned after each use and sanitized daily. Cleaning typically involves washing the components using water and a detergent, and sanitizing typically involves exposing the components to steam. Similarly, baby feeding equipment and components, including baby bottles, that are designed for reuse and used in medical environments, must be cleaned each time they are used. Continuous positive airway pressure (CPAP) therapy components, including tubing, nasal masks, and headgear, should be cleaned in similar manners. However, such tasks require a significant number of components, which can be onerous for users. Accordingly, improved systems and methods for sanitizing breast pump components and CPAP therapy components would be beneficial.

### SUMMARY OF THE INVENTION

In one aspect, the present disclosure provides a system for sanitizing a medical device. The system includes a container having a microwavable sanitization chamber, and the sanitization chamber is configured to receive a liquid and the medical device. A cover is configured to be supported by the container and obscure the sanitization chamber. The cover and the container are configured to be positioned in a microwave and facilitate converting the liquid to steam upon receipt of microwave energy from the microwave, such that the steam sanitizes the medical device. A dryer rack is coupled to the cover, and the dryer rack is configured to support and thereby facilitates drying the medical device.

In another aspect, the present disclosure provides a method for sanitizing a medical device. The method includes positioning the medical device in a sanitization chamber of a microwavable container; partially filling the sanitization chamber with a liquid; positioning a cover on the container and thereby obscuring the sanitization chamber; positioning the container in a microwave while carrying the medical device, the liquid, and the cover; and providing microwave energy to the liquid while positioned in the sanitization chamber and the microwave and thereby converting the liquid to steam, the steam sanitizing the medical device.

### BRIEF DESCRIPTION OF THE DRAWINGS

For illustrative purposes, the drawings show an embodiment which is presently preferred. However, it should be understood that the present disclosure is not limited to the embodiment illustrated in the drawings.

FIG. 1 is a top perspective view of a sanitization system according to an embodiment of the present disclosure; the sanitization system is illustrated in a closed configuration in which a container carries a cover.

FIG. 2 is a top perspective view of the container of the sanitization system of FIG. 1.

FIG. 3 is a bottom perspective view of the cover and a dryer rack of the sanitization system of FIG. 1.

FIG. 4 is a side view of the cover and the dryer rack of FIG. 3.

FIG. 5 is a bottom view of the cover and the dryer rack of FIG. 3.

FIG. 6 is a top perspective view of the sanitization system of FIG. 1 in an open configuration in which the cover is removed from the container, and medical devices are positioned in a sanitization chamber of the container.

FIG. 7 is a top perspective view of the sanitization system of FIG. 1 in the closed configuration after positioning the cover on the container.

FIG. 8 is a top perspective view of the sanitization system of FIG. 1 in the closed configuration and after positioning the system in a microwave.

FIG. 9 is a top perspective view of the sanitization system of FIG. 1 in an open configuration and permitting medical devices to dry on the dryer rack.

FIG. 10 is another top perspective view of the sanitization system of FIG. 1 in the open configuration and permitting medical devices to dry on the dryer rack.

FIG. 11 is a top perspective view of a sanitization system according to another embodiment of the present disclosure; the sanitization system is illustrated in a closed configuration in which a container carries a cover.

FIG. 12 is another top perspective view of a sanitization system of FIG. 11; the sanitization system is illustrated in an open configuration in which the cover is removed from the container.

FIG. 13 is a top perspective view of the container of the sanitization system of FIG. 11.

FIG. 14 is a bottom view of the cover and a dryer rack of the sanitization system of FIG. 11.

FIG. 15 is a top perspective view of the sanitization system of FIG. 11 in an open configuration and permitting medical devices to dry on the dryer rack; the container is also illustrated in a collapsed configuration.

FIG. 16 is a top view of a cleaning brush of the sanitization system of FIG. 11.

FIG. 17 is a top perspective view of a slightly different embodiment of the container of the sanitization system of FIG. 1.

### DETAILED DESCRIPTION OF THE DRAWINGS

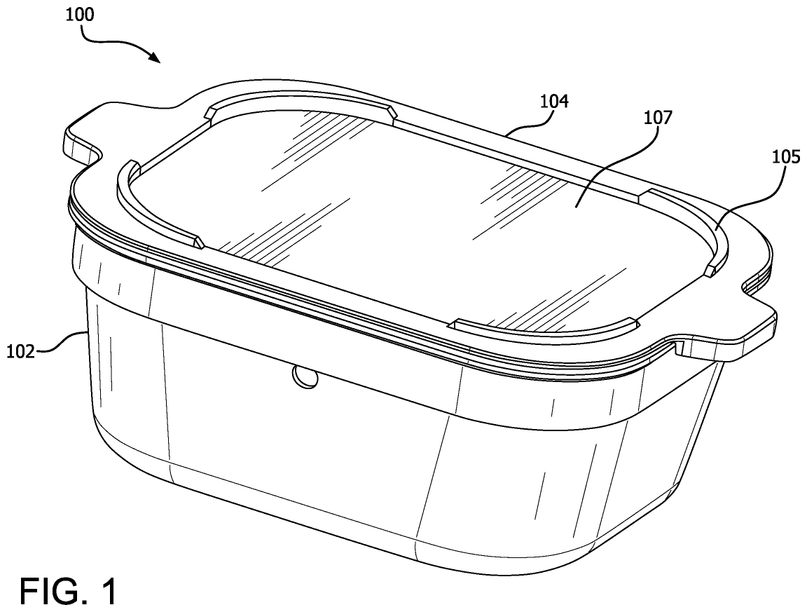

Referring to the drawings, wherein like reference numerals identify corresponding or similar elements throughout the several views, FIG. 1 illustrates a sanitization system 100 according to an embodiment of the present disclosure. The sanitization system 100 generally includes a container 102, which may also be referred to as a basin or a tub, that carries a cover 104 in a closed configuration, as illustrated. The container 102 includes a sanitization chamber (shown elsewhere) that receives and facilitates sanitizing one or more medical components (not shown—for example, one or more human breast milk pump components, baby bottles and reusable feeding equipment, one or more CPAP therapy components, or the like). More specifically, the sanitization chamber facilitates washing one or more medical devices by using a liquid (for example, water) and a detergent (for example, dish, sanitizing or anti-bacterial soap). The sanitization chamber also facilitates sanitizing one or more medical devices by exposing the devices to steam produced by delivering microwave energy to a liquid (for example, water) carried in the chamber. These aspects are described in further detail below.

The sanitization system 100 may be constructed of various microwave-safe materials, such as polymers. In some embodiments and as illustrated, the sanitization system 100 has a generally rectangular profile. Alternatively, the sanitization system 100 may have other a different profile, such as a circular profile. In some embodiments, the sanitization system 100 and the cover 104 have a height in a range of 4.75 in. to 5.25 in., more specifically about 4.9375 in., a length in a range of 11.0 in. to 14.0 in., more specifically about 13.75 in., and a width in a range of 8.0 in. to 9.75 in., more specifically about 9.5625 in.

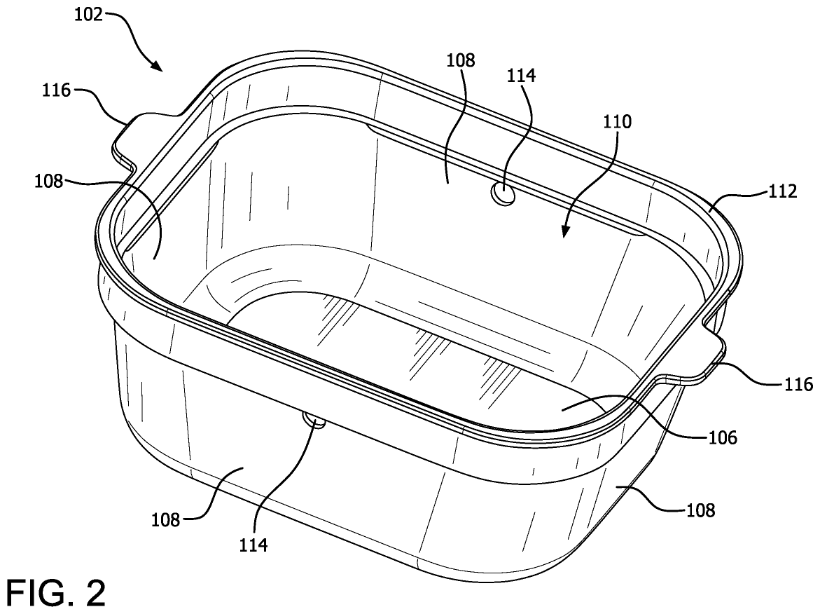

FIG. 2 illustrates the container 102 of the sanitization system 100. The container 102 includes a base 106 and a plurality of walls 108 that define the sanitization chamber 110 and an upper opening 112 coupled the sanitization chamber 110. Although not shown, the container 102 may include feet extending downward from the base 106, preferably in the corners of the container walls so as to space the base 106 above the surface on which it is placed. this facilitates drying and helps limit sliding of the container which might otherwise happen if water is trapped between the base and the surface. The container 102 preferably has a maximum length and width that is similar to but slightly smaller than the cover 104. A sidewall that is slightly tapered facilitates stacking.

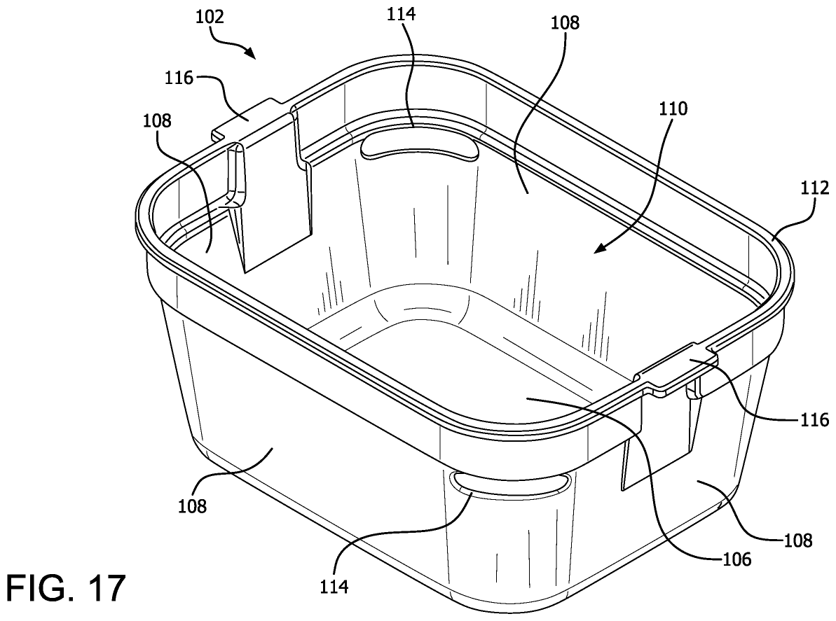

The sanitization chamber 110 also couples to one or more vents 114 formed in the walls 108 (illustratively, two vents 114). The vents 114 can be located in various positions in the sides, such as the corners as shown in FIG. 17. As described in further detail below, the vents 114 facilitate discharging steam from the sanitization chamber 110. In some embodiments, the vents 114 have a diameter of in a range of 1.0 in. to 1.5 in., more specifically about 1.25 in. The vents are designed to relieve pressure inside the chamber that is created by the steam during the sanitizing process (i.e., eh expansion of liquid to gas inside the container during sanitation. The vents are preferably located near the top of the walls 108 or in the cover 104. It is preferable to locate the vents 114 spaced laterally apart from handles 116 so as to reduce the likelihood of steam contacting a person's handles during movement of the container. The number and size are selected so as to maintain a sufficient volume of steam within the chamber in order to sufficiently sanitize the medical devices. In some embodiments, a sufficient volume of steam to sanitize the medical devices may be maintained by providing microwave energy for 3 minutes on a “high” power setting for a typical microwave (that is, the microwave having a power output of 800 W to 1100 W). It should be readily apparent that the amount of time to generate a sufficient volume of steam will vary depending on the container volume size, the power of the microwave and the venting. The walls 108 couple to one or more handles 116 (illustratively, two handles 116). The handles 116 are configured to be grasped by a user and facilitate carrying the sanitization system 100.

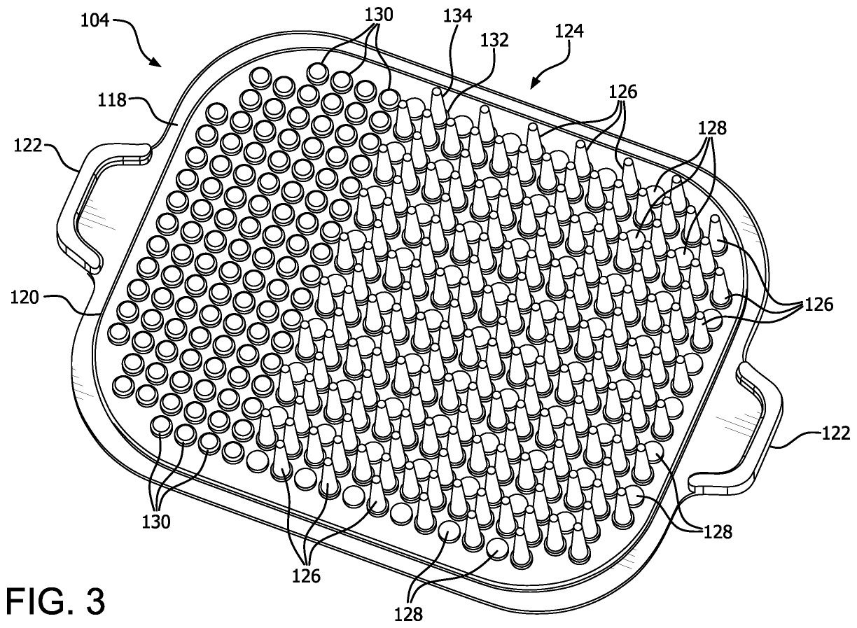

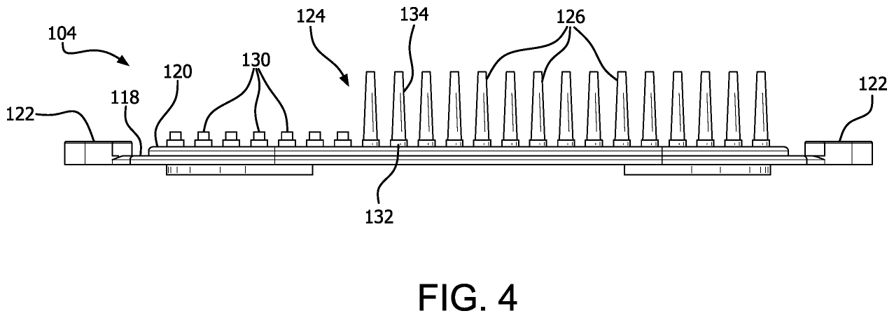

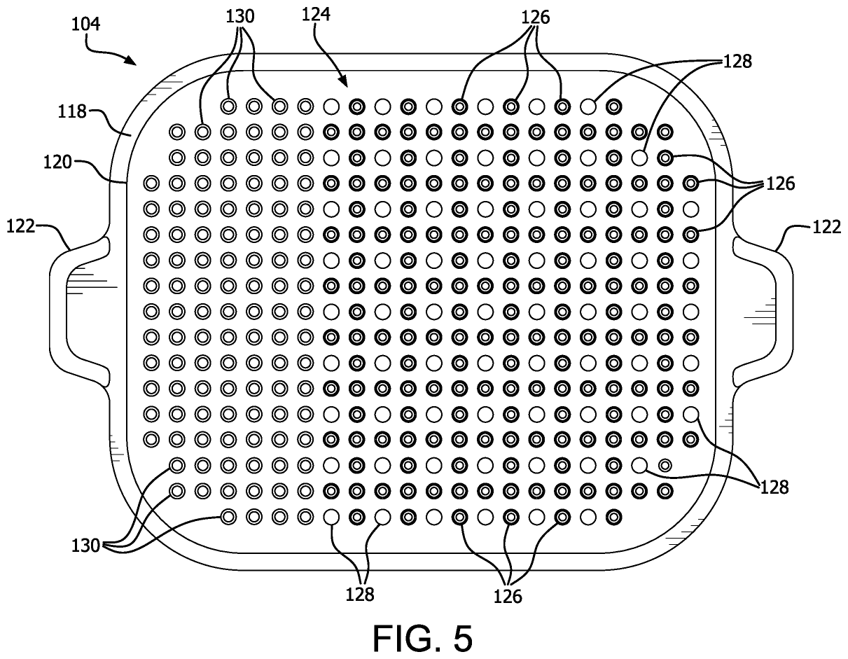

FIGS. 3-5 illustrate the cover 104 of the sanitization system 100. A lower side 118 of the cover 104 includes a protruding surface 120 that is sized to fit into the opening 112 of the container 102. The protruding surface 120 thereby facilitates positioning the cover 104 on the container 102 such that the cover 104 obscures the sanitization chamber 110. The cover 104 includes one or more handles 122 (illustratively, two handles 122). The handles 122 are configured to be grasped by a user and facilitate carrying the sanitization system 100. The handles 122 may be aligned with the handles 116 of the container 102 (shown elsewhere).

With continued reference to FIGS. 3-5, the protruding surface 120 on the lower side 118 of the cover 104 couples to a dryer rack 124. As described in further detail below, the dryer rack 124 may be used to support and facilitate drying medical devices after sanitization. As illustrated, the dryer rack 124 may be monolithically constructed with the cover 104. Alternatively, the dryer rack 124 may be detachably coupled to the cover 104. The dryer rack 124 is preferably configured to be positioned in the sanitization chamber 110 of the container 102 when the cover 104 is supported by the container 102 and obscures the sanitization chamber 110 (that is, when the closed configuration illustrated in FIG. 1).

The dryer rack 124 includes a plurality of supports for supporting medical devices. More specifically, the dryer rack 124 includes a plurality of fingers 126, a plurality of first nubs 128, and a plurality of second nubs 130. As illustrated, the fingers 126 may each include a short cylindrical portion 132 that carries elongated frustoconical portion 134. Alternatively, one or more of the fingers 126 may have different shapes. As illustrated, the first nubs 128 may each have a short cylindrical shape. Alternatively, one or more of the first nubs 128 may have different shapes. As illustrated, the first nubs 128 may be interposed between the fingers 126. Alternatively, the fingers 126 and the first nubs 128 may have different arrangements. As illustrated, each of the fingers 126 may have a first height, each of the first nubs 128 may have a second height, and the second height may be less than the first height. In some embodiments, the first height may be in a range of 1.0 in. to 1.5 in., more specifically about 1.25 in. Alternatively, one or more of the fingers 126 and/or one or more of the first nubs 128 may have different heights. As illustrated, the second nubs 130 are disposed aside the fingers 126 and the first nubs 128. As illustrated, the second nubs 130 may each have a double cylindrical shape, which may also be referred to as a “top hat” shape. As illustrated, each of the second nubs 130 may have a third height, and the third height may be less than the first height of the fingers 126 and greater than the second height of the first nubs 128. Alternatively, one or more of the second nubs 130 may have different heights.

The opposite side of the cover 104 preferably has one or more protruding ribs 105 that extend away from a top surface 107 of the cover. The ribs 105 are configured to space the top surface 107 apart, preferably about 0.5 inches or more, from any surface it is placed upon when it is used as a drying rack as discussed above.

The sanitization system 100 may also include a cleaning brush (not shown) to facilitate washing medical devices by using water and a detergent. The brush may be constructed of various appropriate materials, preferably microwavable-safe, such as polymers.

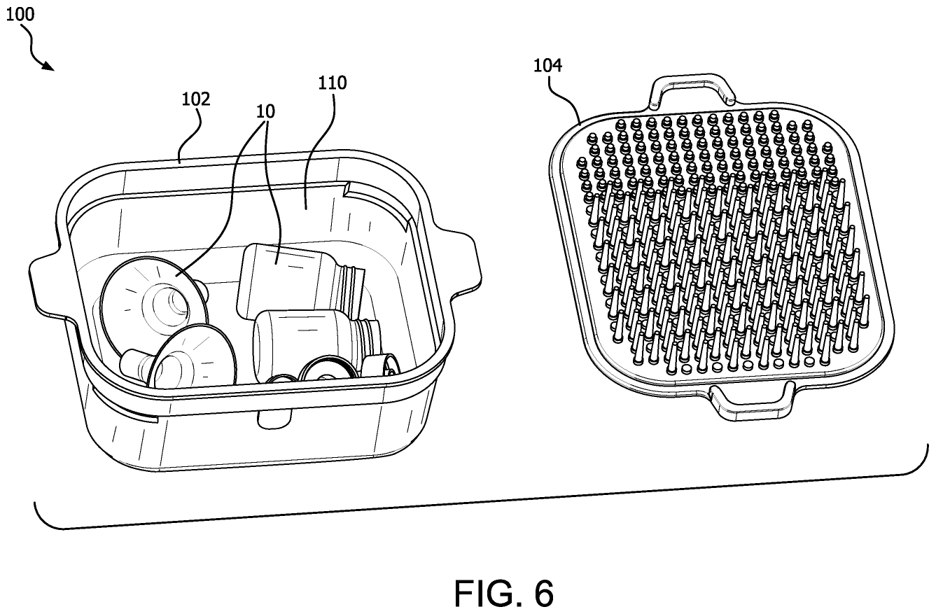

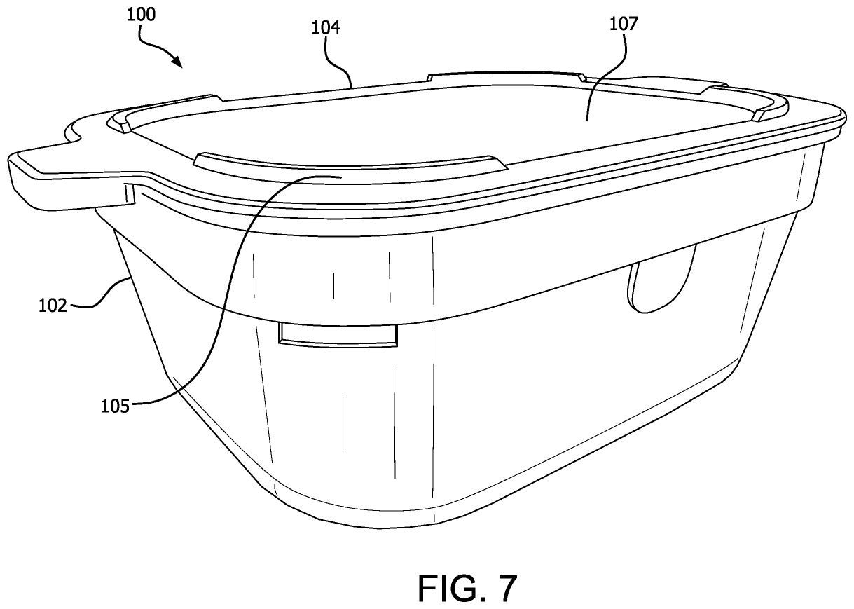

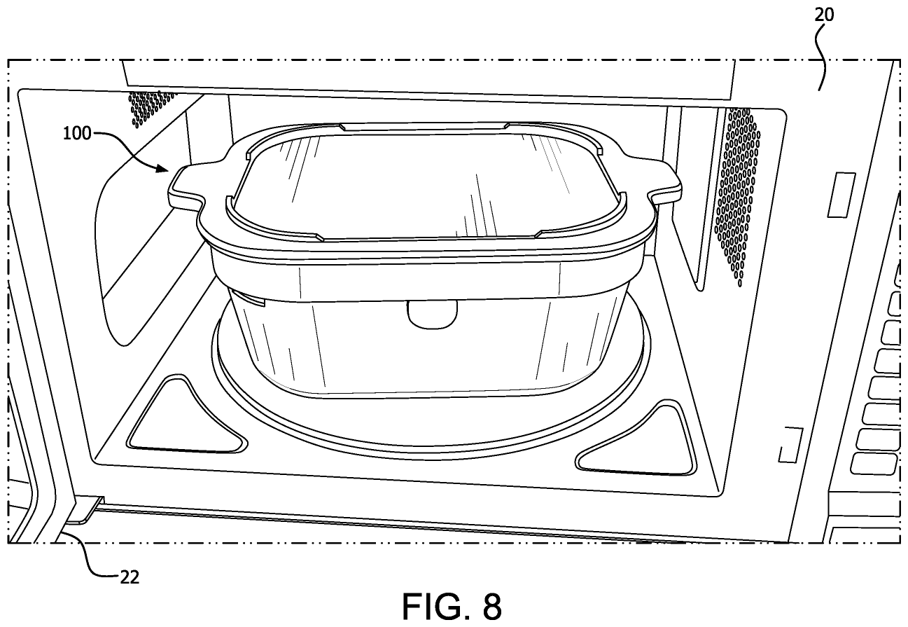

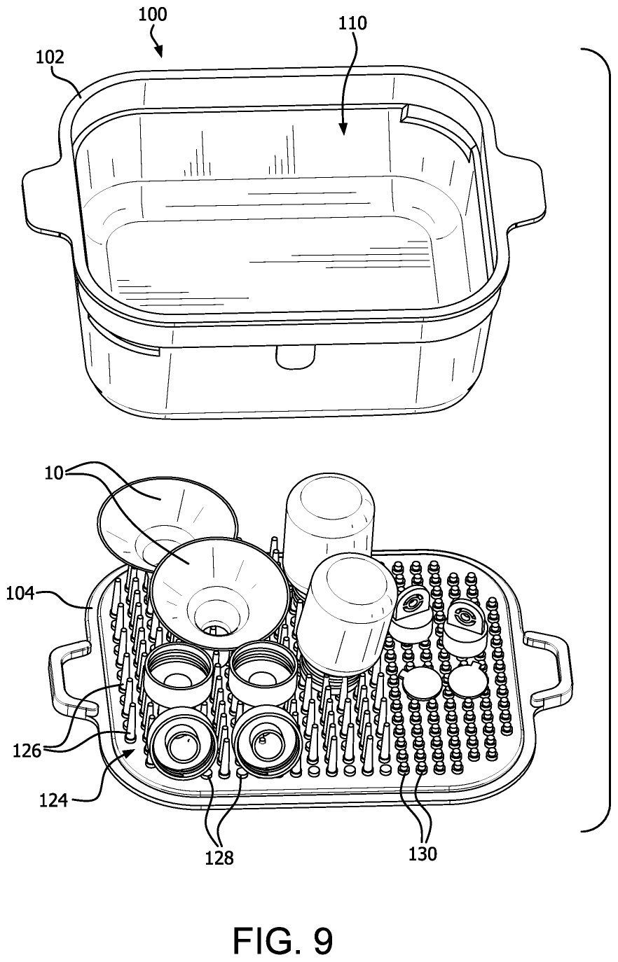

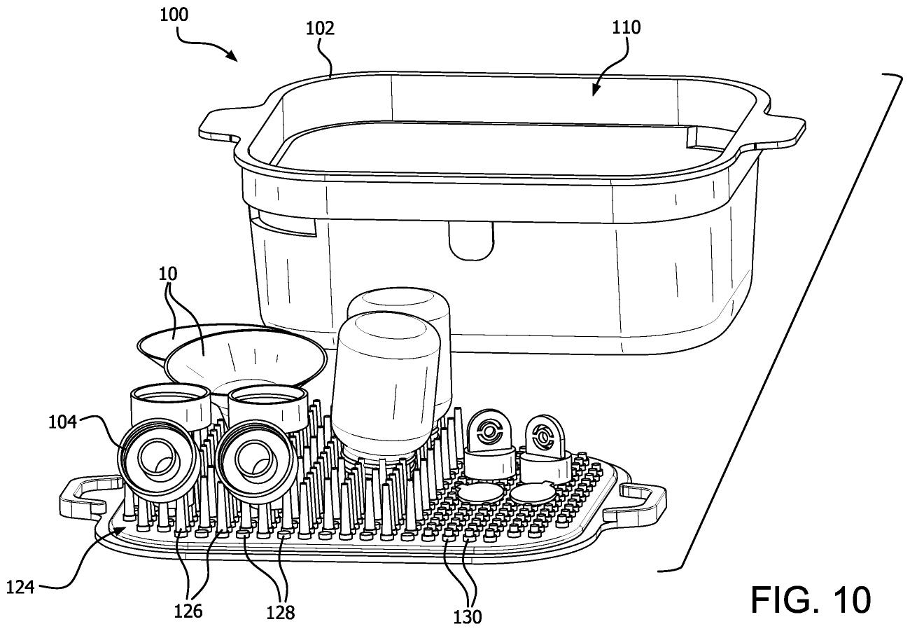

FIGS. 6-10 illustrate a method for using the sanitization system 100 to sanitize one or more medical devices 10 (illustratively, human breast milk pump components). Initially and as shown in FIG. 6, the cover 104 is removed from the container 102 (that is, the sanitization system 100 is configured in an open configuration) and medical devices 10 are positioned in the sanitization chamber 110 of the container 102. Next, the sanitization chamber 110 is partially filled with a liquid (not shown—for example, water). In some embodiments, the sanitization chamber 110 is partially filed with a volume of the liquid in a range of 1 oz. (30 mL) to 3 oz. (90 mL), more specifically a volume of the liquid of about 2 oz. (60 mL). The amount, of course, would vary depending on the volume of the chamber. Then and as shown in FIG. 7, the cover 104 is positioned on the container 102 such that the cover 104 obscures the sanitization chamber 110 (that is, the sanitization system 100 is reconfigured to the closed configuration). Additionally, the cover 104 is positioned such that the dryer rack 124 is positioned in the sanitization chamber 110 of the container 102 together with the medical devices 10. Next and as shown in FIG. 8, the sanitization system 100 is positioned in a microwave 20. Then the door 22 of the microwave 20 is closed, and the microwave 20 is energized to provide microwave energy to the sanitization system 100. More specifically, the microwave 20 may be energized in a range of 800 W to 1100 W for about 3 minutes. Alternatively, the microwave 20 may be energized at a different power level and/or for a different amount of time. Upon receiving microwave energy, the liquid in the sanitization chamber 110 of the container 102 is converted to steam, and the steam contacts and sanitizes the medical devices 10 in the sanitization chamber 110. Next, the sanitization system 100 is removed from the microwave 20. Then and as shown in FIGS. 9 and 10, the cover 104 is removed from the container 102 and positioned such that the dryer rack 124 faces generally upwardly. The medical devices 10 are removed from the sanitization chamber 110 of the container 102 and positioned on the dryer rack 124, specifically the plurality of fingers 126, the plurality of first nubs 128, and/or the plurality of second nubs 130, to facilitate drying the medical devices 10.

As described briefly above, the sanitization system 100 may facilitate washing one or more medical devices by using water and a detergent. More specifically, one or more medical devices may be positioned in the sanitization chamber of the container. Then, the sanitization chamber 110 of the container 102 may be partially filled with a liquid (for example, water) and a detergent (for example, dish soap). Next, the medical devices may be washed using the liquid, detergent, and the brush described above. The medical devices may then be rinsed and removed from the sanitization chamber 110 of the container 102 and positioned on the dryer rack 124 to dry. The sanitization system 100 may be used to wash the medical devices before and/or after sanitizing the medical devices by using the sanitization system 100 to provide steam as described above.

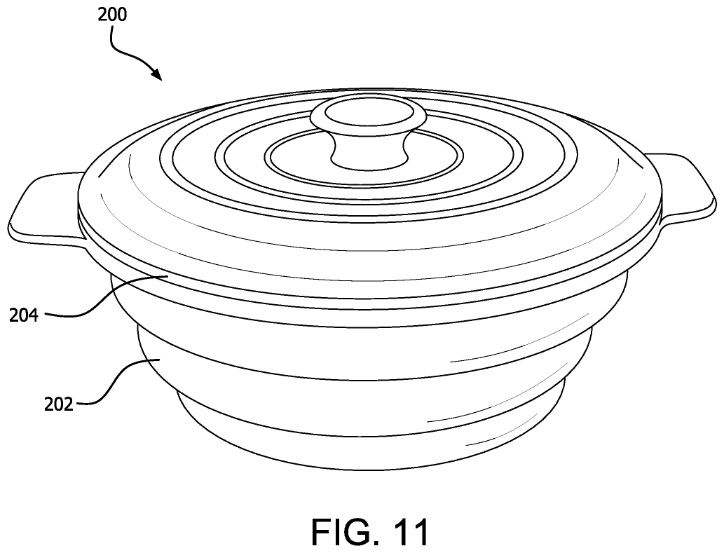

FIG. 11 illustrates a sanitization system 200 according to another embodiment of the present disclosure. The sanitization system 200 is similar to the sanitization system 100 described above. More specifically, the sanitization system 200 generally includes a container 202, which may also be referred to as a basin or a tub, that carries a cover 204 in a closed configuration, as illustrated. The container 202 includes a sanitization chamber that receives and facilitates sanitizing one or more medical components (for example, one or more human breast milk pump components, baby bottles and reusable feeding equipment, one or more CPAP therapy components, or the like). More specifically, the sanitization chamber facilitates washing one or more medical devices and exposing the devices to steam in similar manners to the sanitization system 100 described above.

The sanitization system 200 may be constructed of various microwave-safe materials, such as polymers. In some embodiments and as illustrated, the sanitization system 200 has a generally circular profile. Alternatively, the sanitization system 200 may have other a different profile, such as a rectangular profile.

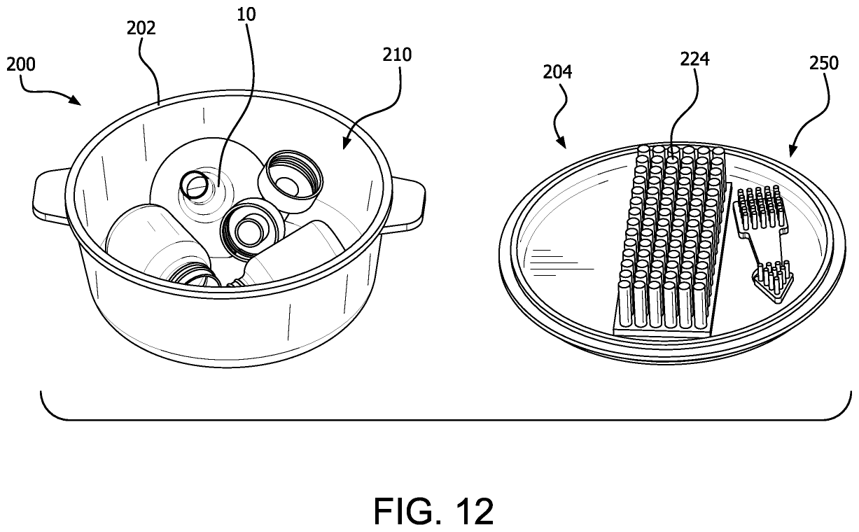

FIG. 12 illustrates the sanitization system 200 in an open configuration in which the cover 204 is removed from the container 202. The container 202 is also illustrated as receiving medical devices 10 (illustratively, human breast milk pump components) in the sanitization chamber 210. The cover 204 is illustrated as including a dryer rack 224 and carrying a cleaning brush 250.

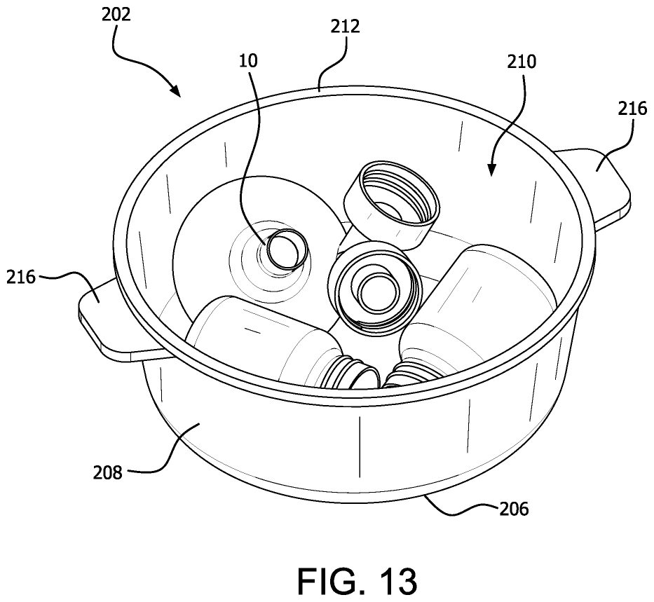

FIG. 13 illustrates the container 202 of the sanitization system 200. The container 202 is illustrated as receiving the medical devices 10 in the sanitization chamber 210. The container 202 includes a base 206 and a wall 208 that define the sanitization chamber 210 and an upper opening 212 coupled the sanitization chamber 210. The wall 208 couple to one or more handles 216 (illustratively, two handles 216). The handles 216 are configured to be grasped by a user and facilitate carrying the sanitization system 200. The wall 208 of the container 202 may be collapsible to facilitate storing the system 200 in a compact form.

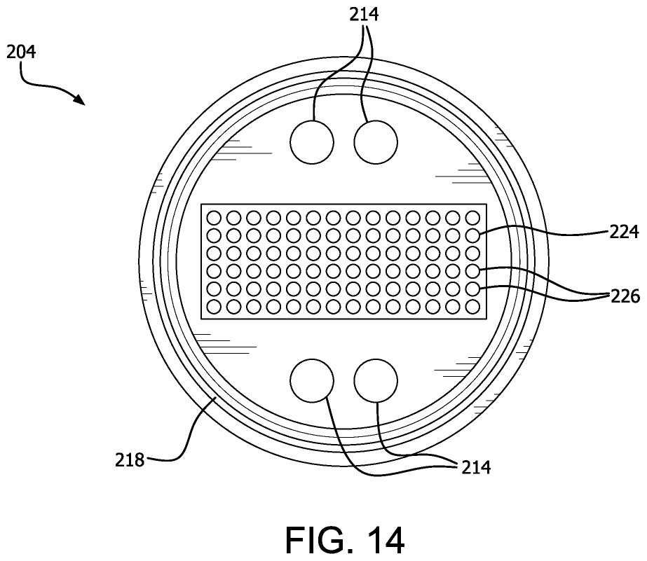

FIG. 14 illustrates the cover 204 of the sanitization system 200. A lower side 218 of the cover 204 couples to the dryer rack 224, which may be used to support and facilitate drying medical devices after sanitization. The dryer rack 224 may be monolithically constructed with the cover 204. Alternatively, the dryer rack 224 may be detachably coupled to the cover 204. The dryer rack 224 includes a plurality of supports for supporting medical devices. More specifically, the dryer rack 224 includes a plurality of fingers 226, each of which may have an elongated cylindrical shape. Alternatively, one or more of the fingers 226 may have different shapes. The dryer rack 224 is configured to be positioned in the sanitization chamber 210 of the container 202 (both shown elsewhere) when the cover 204 is supported by the container 202 and obscures the sanitization chamber 210 (that is, when the closed configuration illustrated in FIG. 11). The cover 204 also includes one or more vents 214. The vents 214 facilitate discharging steam from the sanitization chamber 210 of the container 202 (both shown elsewhere).

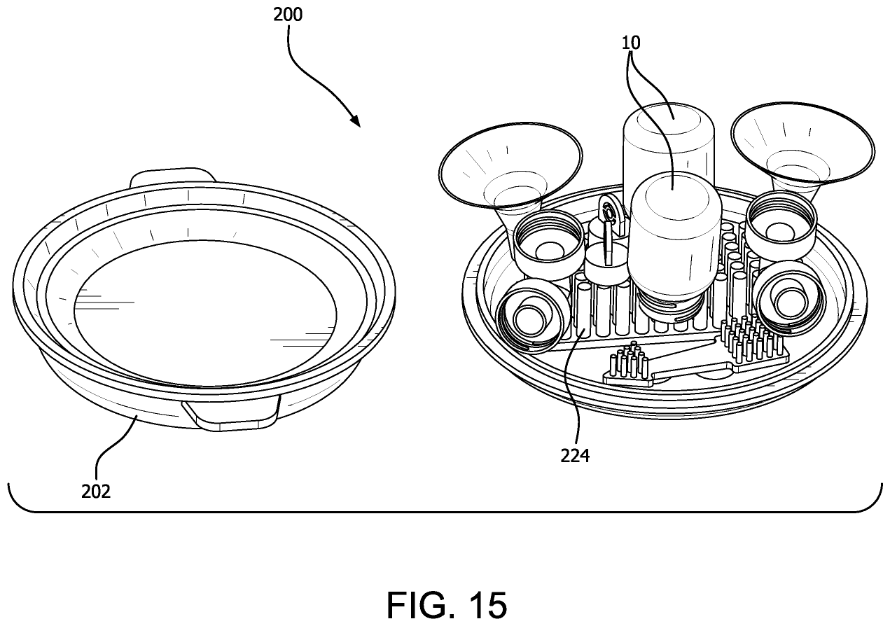

FIG. 15 illustrates the sanitization system 200 in the open configuration and permitting the medical devices 10 to dry on the dryer rack 224 (for example, after using the system 200 to wash the medical devices 10 or expose the medical devices 10 to steam). FIG. 15 also illustrates the container 202 in a collapsed configuration.

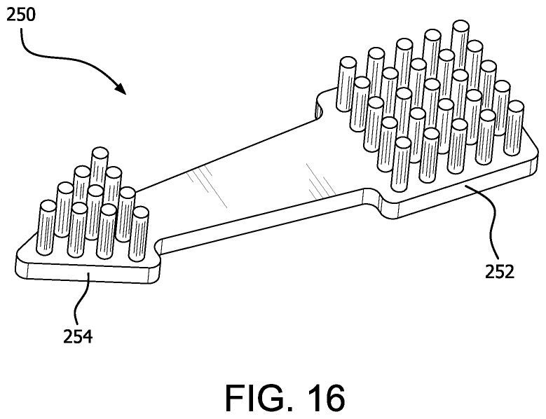

FIG. 16 illustrates the cleaning brush 250 of the sanitization system 200. The cleaning brush 250 includes a flat bristle portion 252 and a tapered cleaning portion 254. The cleaning brush 250 may be constructed of various microwave-safe materials, such as polymers.

While he main intended use for the sanitization system is in hospital or clinical settings, it is also contemplated that the sanitization system can be used at an individual's home. In fact, it is contemplated that a patient will first use the sanitization system in a hospital or clinical office and then take the sanitization system home for continued home use.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The particular implementations shown and described herein are illustrative examples of the invention and are not intended to otherwise limit the scope of the invention in any way. The use of any and all examples, or exemplary language (e.g., “such as”) provided herein, is intended merely to better illuminate the invention and does not pose a limitation on the scope of the invention unless otherwise claimed. Numerous modifications and adaptations will be readily apparent to those skilled in this art without departing from the spirit and scope of the invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening.

The recitation of ranges of values herein are merely intended to serve as a shorthand method of referring individually to each separate value falling within the range, unless otherwise indicated herein, and each separate value is incorporated into the specification as if it were individually recited herein.

Terms such as “about” or “approximately”, unless otherwise defined or restricted in the specification, should be understood to define a variance of plus or minus 5%-10% to the numerical term referred to.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed. The various embodiments and elements can be interchanged or combined in any suitable manner as necessary.

The use of directions, such as forward, rearward, top and bottom, upper and lower are with reference to the embodiments shown in the drawings and, thus, should not be taken as restrictive. Reversing or flipping the embodiments in the drawings would, of course, result in consistent reversal or flipping of the terminology.

No language in the specification should be construed as indicating any non-claimed element as essential to the practice of the invention.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed is:

1. A system for sanitizing a medical device, comprising:
a container made from microwavable-safe material, the container comprising a sanitization chamber, the sanitization chamber configured to receive a liquid and the medical device;
a cover configured to be supported by the container and obscure the sanitization chamber, the cover and the container configured to be positioned in a microwave and facilitate converting the liquid to steam upon receipt of microwave energy from the microwave, the steam sanitizing the medical device; and
a dryer rack coupled to the cover, the dryer rack configured to support and thereby facilitate drying the medical device.

2. The system of claim 1, wherein the dryer rack comprises a plurality of fingers configured to support and thereby facilitate drying the medical device.

3. The system of claim 2, wherein the plurality of fingers comprise a first height, the dryer rack further comprises a plurality of nubs configured to support and thereby facilitate drying the medical device, the plurality of nubs comprise a second height, the second height being less than the first height.

4. The system of claim 2, wherein the dryer rack further comprises a plurality of nubs interposed between the plurality of fingers.

5. The system of claim 1, wherein the dryer rack comprises a plurality of nubs configured to support and thereby facilitate drying the medical device.

6. The system of claim 1, wherein the dryer rack is configured to be positioned in the sanitization chamber.

7. The system of claim 1, wherein the container further comprises a vent coupled to the sanitization chamber.

8. The system of claim 1, wherein the cover further comprises a vent configured to be coupled to the sanitization chamber.

9. The system of claim 1, wherein the container further comprises a handle configured to be grasped by a user.

10. The system of claim 1, wherein the cover further comprises a handle configured to be grasped by a user.

11. A method for sanitizing a medical device, the method comprising:
positioning the medical device in a sanitization chamber of a container;
partially filling the sanitization chamber with a liquid;
positioning a cover on the container and thereby obscuring the sanitization chamber;
positioning the container in a microwave while carrying the medical device, the liquid, and the cover; and
providing microwave energy to the liquid while positioned in the sanitization chamber and the microwave and thereby converting the liquid to steam, the steam sanitizing the medical device.

12. The method of claim 11, further comprising:
removing the medical device from the microwave;
positioning the medical device on a dryer rack; and
permitting the medical device to dry while positioned on the dryer rack.

13. The method of claim 12, wherein the dryer rack is coupled to the cover, and further comprising removing the cover and the dryer rack from the microwave.

14. The method of claim 13, wherein the dryer rack comprises a plurality of fingers, and positioning the medical device on the dryer rack comprises positioning the medical device on the plurality of fingers.

15. The method of claim 11, further comprising:
positioning the medical device in the sanitization chamber of the container;
partially filling the sanitization chamber with a detergent; and
cleaning the medical device in the sanitization chamber by using the detergent.

16. The method of claim 11, wherein the medical device is a human breast milk pump component.

17. The method of claim 11, wherein the medical device is a continuous positive airway pressure therapy component.

18. The method of claim 11, wherein the medical device is a reusable baby feeding component.
